(19) United States
(12) Patent Application Publication
(10) Pub. No.: US 2005/0257287 A1
(43) Pub. Date: Nov. 17, 2005

(54) Vectors capable of imparting herbicide resistance and viral cross protection and methods

(76) Inventors: Amit Gal-On, Ramat Hasharon (IL); Yoel Moshe Shiboleth, Kibbutz Magal (IL); Tsachi Arazi, Kiryat Ono (IL)

Correspondence Address: Mark Friedman;Bill Polkinghorn, Discovery Dispatch, 9003 Florin Way, Upper Marlboro, MD, 20772, US

(21) Appl. No.: 10/466,794

(22) PCT Filed: Jan. 18, 2002
(86) PCT No.: PCT/US02/01299

§ 371 Date: Feb. 11, 2004

(51) Int. Cl. A01H 1/00; C12N 15/82

(52) U.S. Cl. 800/280; 435/468

(57) ABSTRACT

A nucleic acid vector for concurrently imparting herbicide resistance to a plant and cross protecting the plant. The vector includes sufficient potyvirus nucleic acid sequence to permit viral replication and spread. The vector further includes mutations which attenuate symptoms of viral infection in the plant and which abolish transmission of the virus by an insect vector. The vector further includes an additional nucleic acid sequence encoding a protein which imparts resistance to an herbicide when expressed in the infected plant. Further disclosed is a method of concurrently imparting herbicide resistance to a plant and cross protecting the plant against at least one potyvirus comprising inoculating at least a portion of the plant with the vector. Further disclosed are plant cells treated according to the method and virions derived from the vector.

### FIELD AND BACKGROUND OF THE INVENTION

The present invention relates to nucleic acid vectors capable of imparting herbicide resistance and viral cross protection, methods of use thereof and plants expressing same and, more particularly, to vectors based on sequences derived from attenuated potyvirus sequences and further including sequences which impart resistance to a chosen herbicide.

Zucchini yellow mosaic virus is a member of the potyviridae family (Shukla et al. (1989) Adv. Virus Res. 36:273-314). Potyviridae is the largest group of plant viruses and its members infect most commercial or cultivated crops.

Worldwide, ZYMV is one of the most devastating diseases of cucurbit species (e.g., squash, melon, watermelon, cucumber etc.; Desbiez and Lecoq, (1997) Plant Pathol. 46:809-829). As in all potyviruses, the ZYMV genome consists of a single messenger-polarity RNA molecule of about 9.6 kb, encapsidated by ˜2000 units of coat protein (CP), forming a helical, flexuous, filamentous particle of about 750 nm long and 11 nm wide (Desbiez, and Lecoq, (1997) Plant Pathol. 46:809-829 and Lisa et al. (1981) Phytopathology 71:667-672).

Means for attenuating potyviruses in general, and ZYMV in particular, have been described in WO 99/51749 and in Gal-On (2000, Phytopathology 90:467-473). However, these earlier teachings contain neither a hint nor a suggestion that a single vector might be employed to concurrently cross protect a plant and render the plant resistant to an herbicide.

U.S. Pat. No. 5,958,422 as well as WO9602649 and WO9218618 teach modified plant viruses as vectors for heterologous peptides, including peptides useful for vaccination. However, these patents relate to use of plants as bioreactors for vaccine production and do not teach cross protection of the plants themselves. Further, these patents do not teach introduction of herbicide resistance genes into the plants. While use of Glufosinate resistance genes in commercial agriculture is well known (e.g. AgrEvo's Liberty-Link-Oilseed Rape, -Canola, -Maize etc.), this resistance has typically been accomplished by germ line transformation of plants. Such germ line transformation raises concerns about unwanted spread of herbicide resistant plants and or transfer of the herbicide resistance gene to wild plant relatives via pollination (Quist D. and Chapela I. H. (2001) Nature 414(6863): 541-3.)

Whitham et al. (Proc. Natl. Acad. Sci. USA (1999) 96: 772-777) teaches use of a potyvirus expressing an herbicide resistance gene in plants in order to identify plant mutants that affect viral replication, cell to cell and long distance movement within a plant. The teachings of Whitham preclude use of attenuated strains of virus to impart cross protection against subsequent wild type viral infection. In addition, use of the teachings of Whitham in commercial agriculture is infeasible because of the pathogenic outcome of viral infection on agriculturally important plants and the high probability of transmission by insect vectors in the field.

U.S. Pat. No. 6,303,848 to Kumagai et al. teaches use of nucleic acid vectors to impart herbicide resistance to crops using a tobamovirus vector. The teachings of Kumagai require use of a subgenomic plant viral promoter which precludes application of his teachings to potyvirus. Further, the teachings of Kumagai do not include expression of a phosphinothricin acetyltransferase gene which confers resistance to glufosinate ammonium based herbicides. Further, Kumagai teaches use of a tobamavirus which is devastating to plants so that its use in commercial agriculture is infeasible. Further, Kumagai does not teach mutants which are impaired in their ability to be transmitted from plant to plant by their normal mode of transmission i.e. mechanically via infected tissue and contaminated soil. Thus spread of viral vectors according to the teachings of Kumagai cannot be controlled increasing the likelihood of uncontrolled infection in untreated plants.

U.S. Pat. No. 5,766,885 to Carrington et al. teaches expression of foreign genes in a potyvirus vector. However, Carrington fails to teach use of attenuated strains of potyvirus in order to minimize viral symptoms. Further, Carrington fails to teach use of potyvirus vectors to impart herbicide resistance. The scope of the teachings of Carrington is limited to use of plants as bioreactors and added value agricultural traits are not found in his teachings.

There is thus a widely recognized need for, and it would be highly advantageous to have, non-insect transmissible nucleic acid vectors capable of concurrently imparting herbicide resistance and cross protection and, methods of use thereof and plants expressing same devoid of the above limitations.

### SUMMARY OF THE INVENTION

According to one aspect of the present invention there is provided a nucleic acid vector for concurrently imparting herbicide resistance to a plant and cross protecting the plant. The vector includes: (a) sufficient potyvirus nucleic acid sequence to permit a potyvirus to replicate and spread within the plant infected by the vector; (b) a first mutation in the potyvirus nucleic acid sequence which attenuates symptoms of the potyvirus in the plant infected by the vector; (c) a second mutation in the potyvirus nucleic acid sequence which abolishes transmission of the potyvirus by an insect vector; and (d) an additional nucleic acid sequence encoding a protein which imparts resistance to an herbicide when expressed in the plant infected by the vector.

According to another aspect of the present invention there is provided a method of concurrently imparting herbicide resistance to a plant and cross protecting the plant against at least one potyvirus and. The method includes inoculating at least a portion of the plant with a vector including; (a) sufficient potyvirus nucleic acid sequence to permit a potyvirus to replicate and spread within the plant infected by the vector; (b) a first mutation in the potyvirus nucleic acid sequence which attenuates symptoms of the potyvirus in the plant infected by the vector; (c) a second mutation in the potyvirus nucleic acid sequence which eliminates transmission of the potyvirus by an insect vector; and (d) an additional nucleic acid sequence encoding a protein which imparts resistance to an herbicide when expressed in the plant infected by the vector.

According to further features in preferred embodiments of the invention described below, the first mutation includes an amino acid substitution in the HC-Pro gene (SEQ ID NO.: 4) of the conserved FRNK box of the potyvirus nucleic acid sequence.

According to still further features in the described preferred embodiments the amino acid substitution in the HC-Pro gene of the conserved FRNK box of the potyvirus nucleic acid sequence includes a substitution of an Arg to Ile at position 180 within the HC-pro gene product (SEQ ID NO.: 5) of the potyvirus.

According to still further features in the described preferred embodiments the insect vector is an aphid.

According to still further features in the described preferred embodiments the sufficient potyvirus nucleic acid sequence is derived from zucchini yellow mosaic virus (ZYMV).

According to still further features in the described preferred embodiments the additional nucleic acid sequence is at least a portion of a phosphinothricin acetyltransferase coding sequence.

According to still further features in the described preferred embodiments the protein is at least a functional portion of a phosphinothricin acetyltransferase.

According to still further features in the described preferred embodiments at least a portion of a plant treated according to the claimed method is an integral part of the invention, as are progeny of at least a portion of a plant treated according to the method.

According to still further features in the described preferred embodiments an infectious virion harvested from a plant treated according to the claimed method is an integral part of the invention.

The present invention successfully addresses the shortcomings of the presently known configurations by providing a vector for concurrently imparting herbicide resistance and cross protection against wild type virus. Concurrent receipt of these two effects from a single treatment contributes to increased crop yield and significantly reduces production costs. Reduction in production costs stein from both elimination of crop damage and from ease of introducing these traits into a wide variety of commercial plant strains.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a-d depict construction and use of a vector according to the present invention. FIG. 1a is a schematic representation of the AGII genome. AGII non-coding (shaded), coding (open boxes) regions, and the bar gene (bar) are shown. Arrows indicate NIa protease involved in proteolysis of the bar gene product. NIa cleavage sites are indicated by /. Amino acid sequences corresponding to the NIa protease recognition motif are indicated in bold. The termini of the bar protein sequence are indicated by italics. FIG. 1b depicts an RT-PCR analysis of AGII-Bar viral progeny RNAs. Total RNA was extracted from leaves of AGII-Bar and AGII systemically infected plants or from virus-free plants, and subjected to RT-PCR with primers flanking the bar gene insertion point. Positions of RT-PCR primers relative to AGII-Bar genome are shown schematically below. The expected size (bp) of the fragment with (1025) or without (476) the bar gene is marked by an arrow. HindIII-EcoRI-digested Lambda DNA was used as a molecular weight marker (M). FIG. 1c is a histogram illustrating accumulation of AGII-Bar and AGII viruses in systemically infected squash 14 and 26 dpi. The level of each virus was determined by quantitative DAS-ELISA, and is the average of three independent samples taken from three different plants. FIG. 1d shows representative leaves of squash, systemically infected with AGII-Bar or AGII four days after spraying with 0.25% Basta herbicide.

FIGS. 2a-c demonstrate that functional expression of bar via a vector according to the present invention confers resistance to glufosinate ammonium in-planta. FIGS. 2a and 2b illustrate Squash (2a) and melon (2b) plants inoculated with either AGII-Bar (left pots) or AGII (right pots) and sprayed to runoff with 0.5% Basta at 24 or 10 dpi respectively. FIG. 2c shows greenhouse grown cucumbers inoculated with AGII or AGII-Bar (as indicated) sprayed to runoff with 1% Basta at about 45 dpi. Days 0 and 10 indicate time after spraying. FIG. 2d shows hydroponically grown squash inoculated with either AGII-Bar (left pot) or AGII (right pot). Basta (1%) was added in the water, 1 week after inoculation. Photographs were taken 7 days after Basta treatment.

FIGS. 3a-d illustrate Basta resistance of a variety of cucurbit species inoculated with a vector according to the present invention in the field. Watermelon Seedless (FIG. 3a), Melon Ananas-type (FIG. 3b), squash (FIG. 3c), and cucumber (FIG. 3d) were infected with AGII (right panel) or with AGII-Bar (left panel). Plants were sprayed 14 days after planting with 0.5% Basta. Pictures of representative plants were taken 5 days after spraying.

FIGS. 4a-c depict resistance of melons affected with a vector according to the present invention to herbicide in the field. FIG. 4a shows melons (Ananas-type) infected with AGII (right panel) or with AGII-Bar (left panel). Plants were sprayed with 0.5% Basta 14 days after planting. Pictures of representative plants were taken 5 days after spraying. FIGS. 4b and 4c depict weed eradication in a field of Galia-type melons inoculated with a vector according to the present invention. Photographs were taken 5 days (FIG. 4b) and 18 days (FIG. 4c) after spraying, with the indicated concentration of Basta or with water.

FIGS. 5a-c are histograms illustrating the effect of practice of a method according to the present invention on crop yield and fruit number. Data are the averages of 13 plants per treatment. FIG. 5a shows numbers of squash fruit per plant collected during an 18-day period as a function of applied Basta concentration. FIGS. 5b and 5c show the effect of increasing Basta concentration on Ananas and Galia type Melons inoculated with a vector according to the present invention. After 18 days of Basta treatment, potentially marketable sized fruit was weighed (5b) and all fruit was counted (5c). The percentage of Basta sprayed is indicated under each column.

FIGS. 6a-d illustrate viral cross protection in squash with a vector according to the present invention. 6a shows leaves infected with a virulent strain of ZYMV (ZYMV-JV); 6b shows leaves not infected with virus (H); 6c shows leaves inoculated first with the attenuated ZYMV-AGII and then with the virulent ZYMV-JV. 6d shows leaves inoculated first with the attenuated ZYMV-AGII carrying the heterologous human interferon alpha 2a (IFN) gene and then with the virulent ZYMV-JV.

FIG. 7 illustrates, by RT-PCR analysis, that ZYMV-AG carrying a foreign gene (IFN), prevents virulent ZYMV-JV RNA accumulation in Squash. Total RNA was extracted from leaves of squash plants inoculated with indicated viruses 28 days post ZYMV-JV challenge, and subjected to RT-PCR with primers flanking the IFN insertion point. pAGII and pIFN plasmids containing AGII and IFN cDNAs respectively. Mix-reaction mixture devoid of RNA. The expected size (bp) of the fragment with (955) or without (476) the IFN gene is marked by an arrow. HindIII-EcoRI-digested Lambda DNA was used as a molecular weight marker (M).

### DESCRIPTION OF THE PREFERRED EMBODIMENTS

The present invention is of nucleic acid vectors capable of concurrently imparting herbicide resistance and viral cross protection, methods of use thereof and plants inoculated with same which can be used in commercial agriculture.

Specifically, the present invention can be used to impart herbicide resistance to a crop while concurrently affording viral cross protection to the crop.

The principles and operation of nucleic acid vectors capable of imparting herbicide resistance and viral cross protection, methods of use thereof and plants inoculated with same according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For purposes of this specification and the accompanying claims, the terms “cross protection” and “cross protecting” refer to inoculation of a plant with an attenuated strain of a virus in order to impart protection against subsequent infection with a wild type strain of the same virus. Typically, cross protection protects more than 50% of the plants inoculated with the attenuated virus. This definition is essentially as described by H. Lecoq in “Control of plant virus diseases by cross protection” (Plant Virus Disease Control (1998) Hadidi A., Khetarpal R. K. and Koganezawa H. Eds., APS Press)

For purposes of this specification and the accompanying claims, the term “inoculate” refers to any administration of a virally derived material which results in infection of a plant. Thus, inoculation may refer equally to administration of vector nucleic acid and to administration of virions.

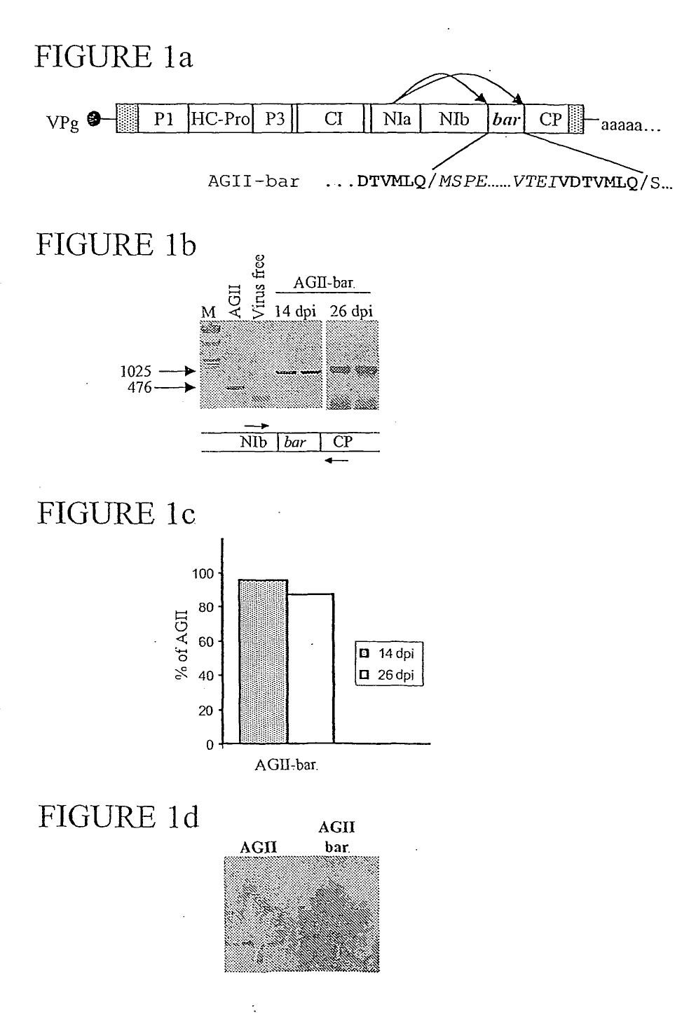

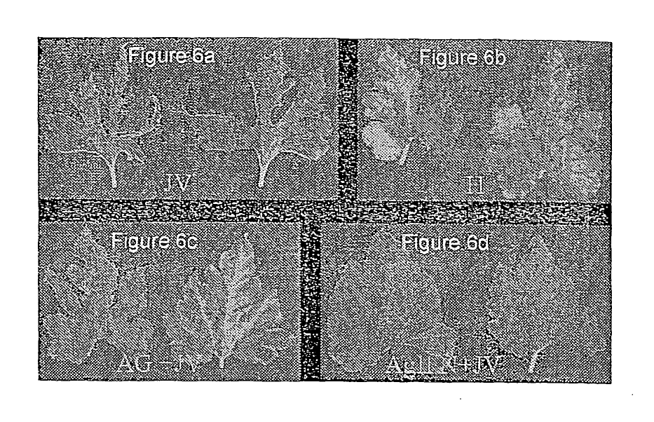

FIG. 1a illustrates an example of a nucleic acid vector according to the present invention. These nucleic acid vectors are designed and constructed to concurrently impart herbicide resistance to a plant and cross protect the plant and. For purposes of this specification and the accompanying claims, the terms “concurrent” and “concurrently” refer to results which share a common causative event. Thus, herbicide resistance may occur before or after appearance of viral cross protection in a plant and the two traits may still be deemed to have been “concurrently imparted” if they are the result of inoculation with the same vector or virus. The vector includes sufficient potyvirus nucleic acid sequence to permit a potyvirus to replicate and spread within the plant infected by the vector. Preferably, the sufficient potyvirus nucleic acid sequence is derived from zucchini yellow mosaic virus (ZYMV). Vectors according to the present invention are infectious and accumulate titers similar to AGII (FIG. 1c), although they are attenuated almost to the point of being asymptomatic (FIG. 6).

The vector further includes a first mutation in the potyvirus nucleic acid sequence which attenuates symptoms of the potyvirus in the plant infected by the vector. The first mutation may include, for example, an amino acid substitution in the HC-Pro gene (SEQ ID NO.: 4) of the conserved FRNK box of the potyvirus nucleic acid sequence. The amino acid substitution in the HC-Pro gene of the conserved FRNK box of the potyvirus nucleic acid sequence may be, for example, a substitution of a Arg to Ile at position 180 within the HC-pro gene product (SEQ ID NO.: 5) of the potyvirus.

The vector further includes a second mutation in the potyvirus nucleic acid sequence which abolishes transmission of the potyvirus by an insect vector (Gal-On et al. (1992) J Gen Virol. 73: 2183-2187).

According to preferred embodiments of the invention, the second mutation includes an alteration of a conserved DAG triplet at position 8 in the coat protein (SEQ ID NOs.: 2 and 3) of the potyvirus. This alteration of the DAG preferably includes a substitution for an alanine residue in the DAG triplet. Alternately, the second mutation may include deletion of the DAG triplet altogether. This may be accomplished, for example, by removal or replacement of the Coat Protein N-terminal domain or a portion thereof (Arazi, T., et al. (2001) J Virol. 75(14): 6329-36). Preferably, the insect vector is an aphid.

The vector further includes additional nucleic acid sequence encoding a protein which imparts resistance to an herbicide when expressed in the plant infected by the vector. Preferably the additional nucleic acid sequence is at least a portion of a phosphinothricin acetyltransferase coding sequence and the protein is at least a functional portion of a phosphinothricin acetyltransferase.

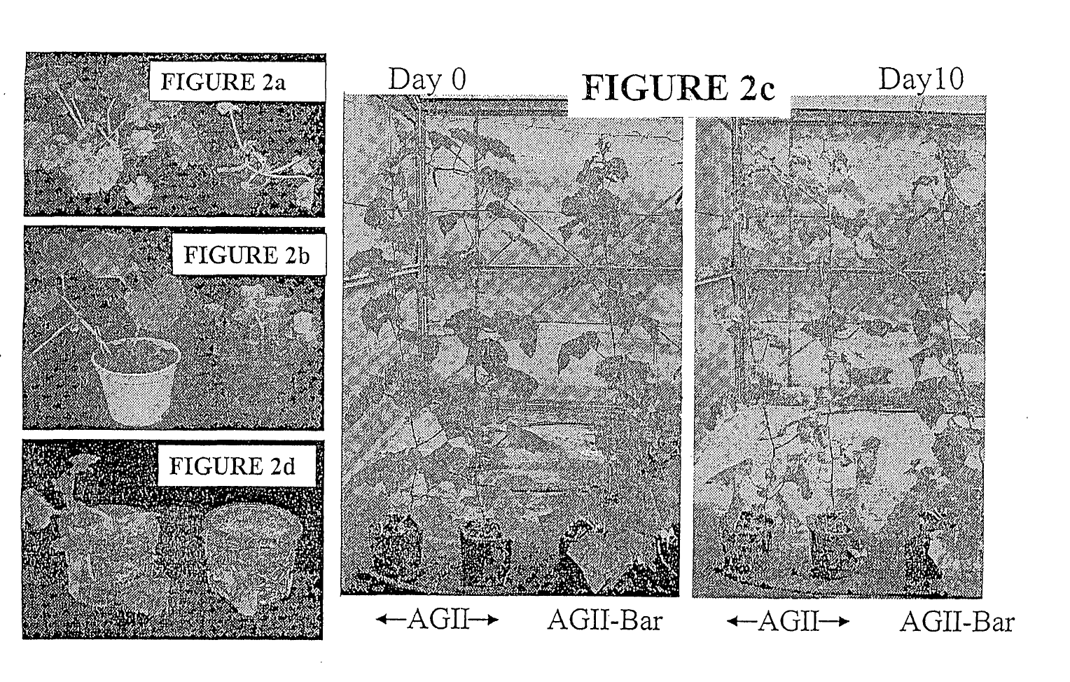

According to another aspect of the present invention there is provided a method of concurrently imparting herbicide resistance to a plant (FIGS. 2, 3 and 4) and cross protecting the plant (FIG. 6) against at least one potyvirus. The method includes inoculating at least a portion of the plant with a vector as described hereinabove

As used in this specification and the accompanying claims, the phrase “at least a portion of the plant” may include, but is not limited to, at least one cell in the plant, at least one plant cell in tissue culture.

The claimed invention is further embodied by at least a portion of a plant treated according to the claimed method. Progeny of at least a portion of a plant treated according to the method are also within the scope of the claimed invention, as are infectious virions harvested from a plant treated according to the claimed method.

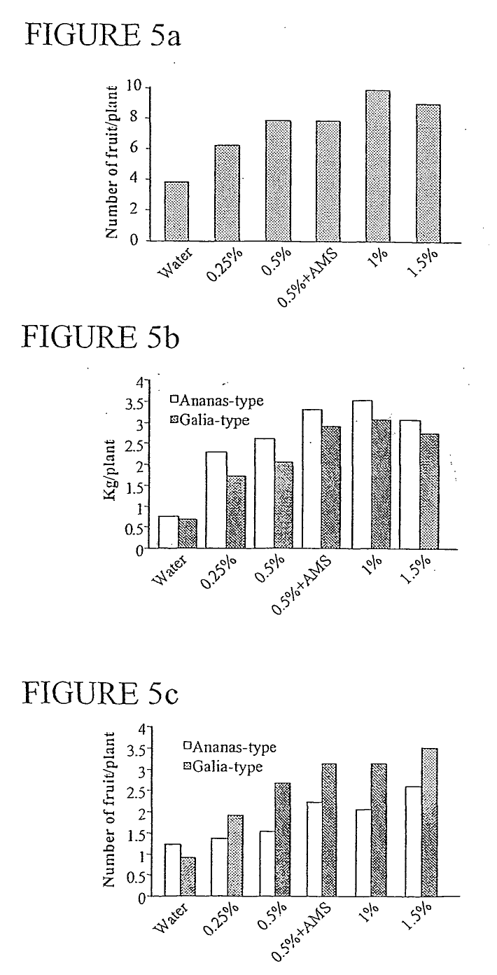

It will be appreciated that, prior to the advent of the claimed invention, cross protection was not generally practiced in commercial agriculture. However, because the claimed invention offers prevention of yield loss by both herbicide resistance for weed eradication in the field (FIG. 5) and prevention of yield loss from viral infection in a single application, it is likely to increase the prevalence of viral cross protection in commercial settings. Together, these features offer unprecedented increases in crop yield. This is because both viruses and weeds are major sources of economic loss in commercial production of cucurbit crops.

Further, the herbicide resistance conferring and cross protecting vectors of the present invention may readily be used to infect many different species and cultivars without the costly and time consuming process of generating transgenic or transformed plants. Therefore, any new cultivar on the market can be protected without additional breeding, seed collection and screening. Protection may be induced, for example, before planting or in the field.

Further, vectors of the present invention are not germ line incorporated. Therefore, maximum selection pressure may be applied to desired traits without including selection for vector-born traits in the breeding plan.

Further, vectors according to the present invention are not seed or pollen transmitted. This insures that the recombinant nucleic acid of the vector is not accidentally transmitted to progeny or to other plant species. This feature addresses issues of containment, which have slowed development of many plant biotechnology products.

Further, vectors according to the present invention overcome the major problems previously associated with potyvirus vectors, i.e. loss due to disease and uncontrolled spread by insect vectors.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non-limiting fashion.

The following materials and methods were employed in executing the experiments described in the examples presented hereinbelow.

### Materials and Methods

AGII-Bar Construction

To construct AGII-bar, the bar gene (GenBank acc. nr. X17220) was amplified from pME509, by using a Taq polymerase and the oligonucleotide primers: sense 5′-ATGCTGCAGATGAGCCCAGAACGACGC-3′ (SEQ ID NO.: 7) and antisense 5′-AGTCTCGAGGATCTCGGTGACGGGCAG-3′ (SEQ ID NO.: 8) which added PstI and XhoI sites (underlined) to the 5′ and 3′ ends of the bar sequence respectively. The amplified fragment was double digested by PstI and XhoI and cloned into the PstI and SalI sites of the partial clone pKSΔSacI-PstI-poly (Arazi et al. (2001) Journal of Biotechnology 92:37-46). pKSΔSacI-PstI-poly-Bar clone was double-digested by SacI and MluI, and the resulting fragment containing the bar gene were cloned into the AGII genome (Arazi et al. (2001) Journal of Biotechnology 92:37-46) between the coat protein (CP) and the NIb coding regions, to create AGII-Bar (FIG. 1).

Plant Growth, Inoculation, and Evaluation of Virus Infection

Potted squash and melon plants were grown in a growth chamber under continuous light at 23 degrees C. Cucumbers (cv. Muhassan) were grown in 20 Liter pails with automatic irrigation and fertilization, in an insect-proof net-house. Hydroponically grown squash were seeded in vermiculite that was placed on nylon nets floated in a water-filled container. Particle bombardment with a hand held device, the HandGun (Gal-On et al. (1997) Journal of Virology Methods 64:103-110), was used to propel micro-projectiles containing a plasmid with AGII-Bar cDNA into the fully expanded cotyledons of different cucurbits. Mechanical inoculation of seedlings and enzyme-linked immunosorbent assay (ELISA) with anti-ZYMV CP polyclonal antibody, performed on infected plant material, were performed as described previously by Antignus et al. (1989; Phytoparasitica 17: 289-298).

ELISA Assays for Evaluation of Infectivity and Viral Titer

Double antibody sandwich enzyme-linked immunosorbent assay (DAS-ELISA) with anti-ZYMV CP polyclonal antibody (1:2000), was performed on infected plant material, as described previously by Antignus et al. (1989; Phytoparasitica 17, 289-298). For quantitative analysis, three squash plants from each treatment, AGII and AGII-Bar were chosen. Eight leaf discs per plant were taken from two different leaves 14 and 26 days post inoculation (dpi), combined, and the homogenized tissue samples were subjected to DAS-ELISA. All samples were collected from developmentally equivalent leaves at the indicated dpi. The significance of the differences in the accumulation of the AGII and AGII-Bar was determined by ANOVA statistical analysis with Statview statistical software package.

RT-PCR Analysis of Recombinant Virus Progeny

RT-PCR of viral progeny was conducted in a one-tube single-step method modified from Seliner et al. (1992; Nucleic Acids Research 20:1487-1490). Briefly, a 50-μl volume was used containing the polylinker flanking primers 5′-AGCTCCATACATAGCTGAGACA-3′ (SEQ ID NO.: 9) and 5′-TGGTTGAACCAAGAGGCGAA-3′ (SEQ ID NO.: 10) in the following mixture: 1.5 mM MgCl₂; 125 μM dNTPs; 1× Sellner buffer: [10× Sellner buffer contains: 670 mM Tris-HCl; 170 mM (NH₄)₂SO₄; 10 mM beta-mercapto-ethanol; 2 mg/ml gelatin (Aldrich, calf skin 225 bloom); 60 μM EDTA pH 8.0 (Sellner et al., 1992)]; 100 ng of each specific primer; 2 units of Taq polymerase; 5 units of AMV-RT (Chimerex USA); 2-5 μg total RNA. RT-PCR cycles were as follows: 46 degrees C. 30 min; 94 degrees C. 2 min, followed by 33 cycles at 94 degrees C., 60 degrees C. and 72 degrees C., each of 30 s., and one final cycle of 5 min at 72 degrees C.

Field Experiment

Two varieties of melon (Galia-type cv. 5080 and Ananas-type cv. Ofir), two varieties of watermelon (cvs Crimson and Seedless 313), squash (cv. Maayan) and cucumber (cv. Shimshon) were seeded in Speedling type trays. Seedlings were inoculated by bombardment of cotyledons with the AGII-Bar or AGII construct. Plants were planted in a 500 m² field, in a sandy loam soil, at 0.5 m intervals in six 2 m wide rows and irrigated by a single line of 0.5 m interval dripline. Each row included 15 plants of each of the five-cucurbit varieties, 13 plants inoculated with AGII-Bar and two inoculated with AGII as a control. Irrigation was initiated a week previously to boost weeds and facilitate planting. Rows were sprayed 13 days after planting with a motorized back-mounted sprayer (Solo, Germany), installed with a boom with 4 overlapping (distance 0.5 m, total width 2 m) T-jet 11002 nozzles (Spraying Systems, USA). Spray height was 0.5 m; pressure 40 PSI, with an actual average of 300 L/hectare. Plants were sprayed with different dosages of the glufosinate-ammonium herbicide Basta 20 (AgrEvo, Germany) containing 200 g/L active ingredient. The active ingredient was calculated per hectare (g a.i./ha) on basis of ground speed measured separately for each treatment. Treatments were given per row: “1.5% Basta”—930 g a.i./ha, “1% Basta”—600 g a.i./ha, “0.5% Basta+AMS”—300 g a.i./ha augmented with ammonium sulphate (AMS) 1.5 g/L., “0.5% Basta”—270 g a.i./ha (without AMS), “0.25% Basta”—165 g a.i./ha and finally “Water” as untreated control. Squash fruit were picked and counted daily. Melon fruit were picked once 66 days after planting and were manually sorted into potentially marketable and unmarketable sizes. All AGII-Bar inoculated plants were picked from each of the six treatment rows. Each group was counted and then weighed collectively.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1994); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); and “Using Antibodies: A Laboratory Manual” (Ed Harlow, David Lane eds., Cold Spring Harbor Laboratory Press (1999)) all of which are incorporated by reference as if fully set forth herein. Other general references are provided throughout this document. The procedures therein are believed to be well known in the art and are provided for the convenience of the reader. All the information contained therein is incorporated herein by reference.

### EXAMPLE 1

### Construction of the AGII-Bar Vector

In order to express the phosphinothricin acetyltransferase gene (hereinafter bar gene; SEQ ID NO.: 1) in the AGII virus-vector, the gene was inserted between the NIb (Genbank accession number L29569) and CP (SEQ ID NO.: 2) genes using a polylinker-cloning site next to the NIa proteinase cleavage site in the NIb 3′ end of AGII (FIG. 1a). The inserted gene was designed to create an in-frame translational fusion with the flanking NIa processing sites. Proteolysis of the nascent AGII-Bar polyprotein by NIa protease in trans was predicted to yield the bar gene product (SEQ ID NO.: 6) with seven additional amino acid residues (VDTVMLQ) at its C′-terminus (FIG. 1a; SEQ ID NO.: 6).

### EXAMPLE 2

### Assay of Infectivity of the AGII-Bar Vector

AGII-Bar was 100% infectious on susceptible squash. Symptoms appeared 7-8 days post-inoculation (dpi) with similar characteristics to those of the parental AGII virus. Squash was employed as the test plant because it is the only cucurbit in which the attenuated AGII symptoms are visible. No symptoms at all are seen on cucumber, melon, pumpkin and watermelon. AGII symptoms in squash include slight vein clearing in young leaves and light patches on older leaves. Slight dark patches appear on fruit of light colored varieties of squash. No deformation or filiform leaf appearance characteristic of the wild type ZYMV are visible. Wild type ZYMV infected plants are highly deformed in leaf and fruit, foliar symptoms consisting of a prominent yellow mosaic, necrosis, distortion, and stunting. Fruits remain small, greatly malformed, and green mottled causing total loss of yield.

In sharp contrast, AGII infected plants, whether carrying a foreign gene or not, yield fruit in number, weight and quality equivalent to virus free plants in the field or in the greenhouse.

100% infectivity was also observed in cucumber, melon and watermelon, though no symptoms were visible in these plants. The presence of the intact bar coding sequence was verified by RT-PCR of the viral progeny (FIG. 1b) and direct sequencing of the amplified product. AGII-Bar, accumulated to levels similar to the parental AGII (no significant difference) 14 and 26 dpi in squash, as determined by quantitative DAS-ELISA (FIG. 1c). The resistance of AGII-Bar infected plants to 0.25% Basta treatment was easily discernible (FIG. 1d).

### EXAMPLE 3

### Induction of Gluofosinate Ammonium (Basta) Resistance in Curcurbit Species Using the Vector

The biological activity of the bar gene product translated from the inoculated vector was tested in the greenhouse. Various concentrations of the glufosinate ammonium based herbicide Basta (Hoechst-Schering AgrEvo, Berlin, Germany) were applied to foliage of squash plants mechanically inoculated with AGII or AGII-Bar from second-generation infected plants. 26 dpi, plants were sprayed till runoff with Basta. All sprayed AGII inoculated plants developed widespread necrosis and died in less than 48 Hrs including plants sprayed with the lowest dose used, 0.125% (Table 1). In contrast, all AGII-Bar plants survived and no necrosis was observed on young and newly emerged leaves even at the highest concentration, 1.0% (Table 1). A representative squash plant is shown in FIG. 2a.

Some herbicide mediated necrosis was observed, mainly on cotyledons and on the first 2-3 leaves, and was positively correlated to the Basta concentration (Table 1).

TABLE 1
Responses of potted squash plants inoculated with
AGII-Bar or with AGII to foliar application of glufosinate
ammonium (Basta) in the greenhouse.

| | |
|---|---|
| AGII | AGII-Bar |

| % Basta | Survival ^{a} | Survival | Necrosis index ^{b} |
|---|---|---|---|
| Unsprayed | 3/3 | 5/5 | − |
| 0.125 | 0/3 | 10/10 | + |
| 0.25 | 0/3 | 10/10 | ++ |
| 0.5 | 0/3 | 11/11 | +++ |
| 1.0 | 0/3 | 8/8 | ++++ |

^{a} Survival from total tested plants
^{b} Necrosis on first 2-3 leaves and cotyledons:
− no necrosis or lesions;
+ few and mild lesions;
++ few patchy lesions;
+++ large necrotic areas;
++++ two thirds or more of the leaf area are necrotic.

Similar results were obtained with melon seedlings (FIG. 2b). As squash and melon were found to be resistant to 1.0%, this concentration was tested on a commercial variety of parthenocarpic cucumber. Cucumber seedlings were inoculated with AGII-Bar or AGII and planted in a nethouse. One and a half months post inoculation, when these plants were fully developed and fruiting, they were sprayed with 1.0% Basta. Forty-eight hours after spraying, leaves of AGII inoculated plants were completely shriveled and dry (FIG. 2c, at 10 days after spray). AGII-Bar plants did not sustain observable damage and continued to develop normally (FIG. 2c).

In an effort to discern the extent of protection afforded by the bar gene, squash plants were grown in a hydroponic system and inoculated with AGII-Bar or control AGII construct. Seven days post inoculation 0.2% ammonium glufosinate (1% Basta) was added to the water in which the roots were immersed. Two days later AGII inoculated plants had died whereas AGII-Bar plants continued to develop though some necrosis was observed, especially to older leaves (FIG. 2d).

### EXAMPLE 4

### Assay of Glufosinate Ammonium (Basta) Resistance in the Field

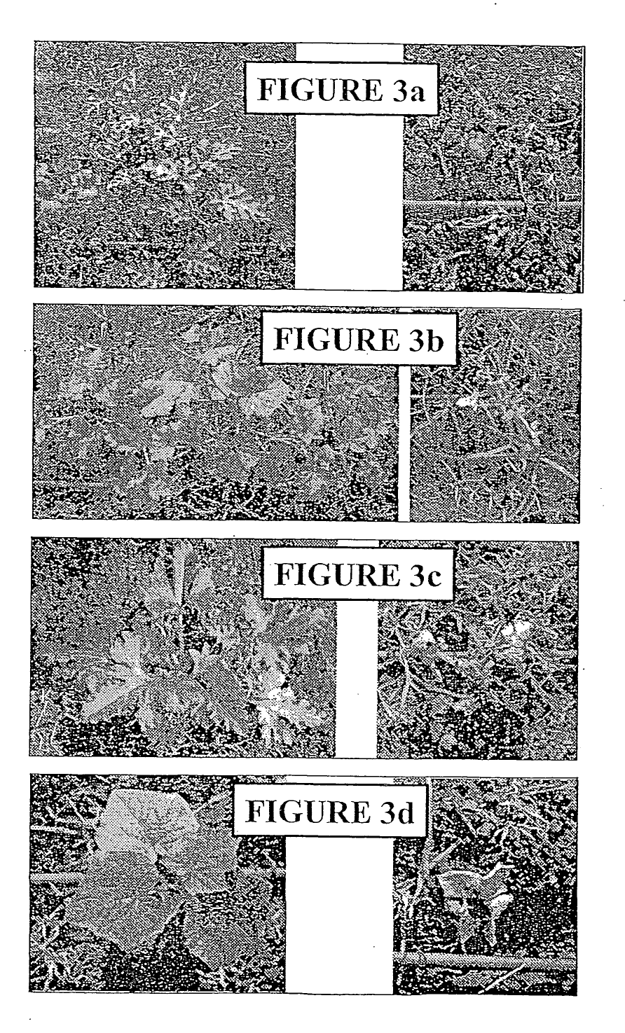

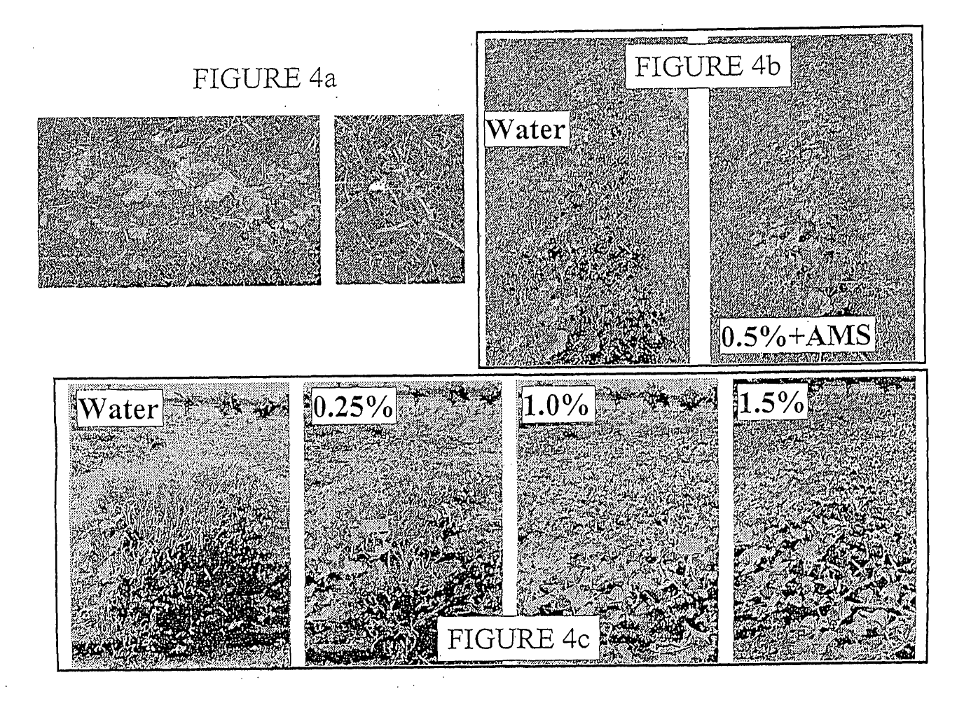

In order to verify the applicability of AGII-Bar to commercial cucurbit cultivation in a field prone to weed infestation an experiment including more than 450 plants was conducted. Plants were inoculated prior to planting by bombardment with AGII-Bar (FIGS. 3a, 3b, 3c and 3d; left hand photograph and FIGS. 4b and 4c), or with AGII (FIGS. 3a, 3b, 3c and 3d; right hand photograph) as a control. Two varieties of melon, two varieties of watermelon, squash and cucumber were planted in the field. The field was pre-irrigated for one week by drippers to increase weed proliferation and facilitate planting. Ten day post inoculation, the infection rate was determined by ELISA of 50 random plant samples and found to be 100%. Two weeks after planting the field was sprayed by rows with different doses of Basta, with or without AMS (FIGS. 4a, 4b and 4c), used as an adjuvant (Maschhoff, J. R., (2000). Weed Sci. 48, 2-6). The control row was sprayed with water. After just 24 hours the damage to AGII inoculated plants and weeds became evident, and in 3-5 days all AGII inoculated plants and most weeds had browned, shriveled and died (FIGS. 4a and 3 a-d; right hand photograph). This response was dose dependent as expected (FIG. 4c).

In sharp contrast, all AGII-Bar inoculated plants survived, although some minor necrotic lesions occurred on the first leaves, at high dosages of Basta. This damage did not adversely affect the vigorous new growth (FIGS. 4a-c and 3a-d). The fact that the weed density in the field would have been high in the absence of herbicide application is demonstrated by the water sprayed rows in Galia type melon (FIGS. 4b and 4c) All weeds including nutgrass (Cyperus rotundus L.), little hogweed (Portulaca oleracea L.), pigweed (Amaranthus sp.) and several annual graminaceous weeds were eliminated in 1-1.5% Basta (FIG. 4c), and most weeds were eliminated or suppressed at 0.25-0.5% as well (FIG. 4c). AMS did not exert a detectable difference on herbicidal activity (FIGS. 4b and c). Herbicide dose-related weed suppression was sustained for the duration of the experiment, 53 days from spray to harvest.

The dose effect of applied glufosinate ammonium on yields of weed-infested fields of squash and of Galia-type and Ananas-type melons was also examined (FIGS. 5a-c). Squash cv. Maayan fruit were picked regularly, collectively by treatment, during an 18-day period. An average count of all thirteen plants picked per row was calculated (FIG. 5a). In the control water-sprayed row, the number of fruit per plant, 3.8, was 1.5-2.5 times less than in 0.25%-1.0% Basta sprayed rows respectively (FIG. 5a). The highest average number of fruit per plant was counted at 1% and 1.5%, 9.8 and 9 fruit respectively.

Melons were picked several days prematurely, 66 days from planting. Potentially marketable yields of 0.25-1.5% Basta sprayed rows, of both melon types, were about 2.5 to more than 4-fold higher than water sprayed control, and showed a positive dose dependant response till a maximum achieved at 1% (FIG. 5b).

As all the melons were picked at once, unselectively, a second parameter for potential yield, fruit number, which included undersized fruits, was assessed. The number of fruit, which had set in the Ananas-type cultivar at the 0.25% Basta treatment (FIG. 5c, white bars), was about the same as the water sprayed control. The highest number of fruit set at the 1.5% Basta treatment, twice as high as the control. However, the number of fruit which had set in the Galia-type cultivar (FIG. 5c, gray bars), which was distinctly positively dose-related, was markedly higher than the water sprayed control, even at the lower concentrations of Basta. In this cultivar, in the 0.25% Basta treatment, twice as many fruit set than in the control, and the number of fruit which set in the 1.5% treatment, which had the highest fruit count of all treatments, was about fourfold higher than the control.

These results confirm the ability of the AGII-Bar vector to impart herbicide resistance determined in Example 2 in the field and further indicate that the imparted herbicide resistance allows application of an herbicide during the production cycle so that crop yield may be significantly increased.

### EXAMPLE 5

### Assay of Recombinant AGII Vector Cross Protection

In order to establish that an attenuated ZYMV vector with impaired aphid transmissibility could impart viral cross protection, and that this ability was not diminished by introduction of a heterologous gene into the vector, 20 squash plants (cv Maayan) were seeded in pots and grown in a green house. Nine days after mechanical pre-inoculation with AGII or AGII-IFN (an AGII construct carrying a foreign gene (human Interferon alpha 2a; Arazi T., et al. (2001) J Biotechnol. 87(1): 67-82.), plants were challenged by mechanical inoculation with wild type virulent ZYMV-JV. Development of symptoms was monitored and recorded (see FIGS. 6 a-d and table 2). Only ZYMV-JV infected leaves are deformed (FIG. 6a). Leaves from plants pre inoculated with AGII (FIG. 6c) or AGII-IFN (FIG. 6d) challenged with ZYMV-JV resembled leaves from virus free plants (FIG. 6b).

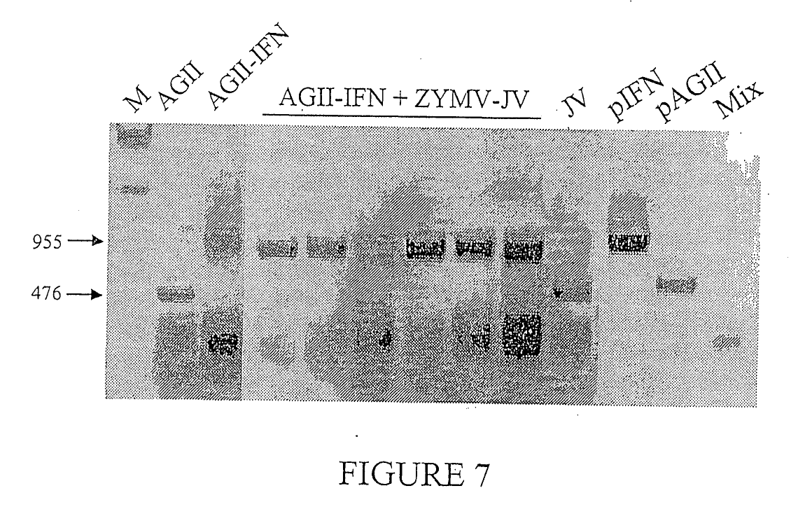

RT-PCR analysis of extracts prepared 28 days post ZYMV-JV challenge from all plants was performed. Results are presented in FIG. 7. AGII or ZYMV-JV infection of plants is expected to yield a 436 bp PCR fragment. The AGII-IFN construct is expected to yield a 955 bp fragment. FIG. 7 clearly demonstrates that all plants pre-inoculated with AGII-IFN were protected from the virulent wild type ZYMV-JV virus (lanes AGII-IFN+ZYMV-JV). These plants showed no disease symptoms (FIG. 6a-d) and AGII-IFN plants had only one PCR band at 955 bp as expected (FIG. 7 and table 2).

TABLE 2
Viral cross protection experiment treatment protocol and results

| | | | | DNA fragment |
|---|---|---|---|---|
| | | | Visible | size (amplified |
| | | | results 26 | by RT-PCR, 28 |
| | Primary | Secondary | days post- | days post- |
| Treatment | inoculation | inoculation | challenge | challenge ) |
| AGII | AGII | No | 2/2 | 436 bp |
| | | | Attenuated | |
| AGII-IFN | AGII-IFN | No | 2/2 | 955 bp |
| | | | Attenuated | |
| AGII + | AGII | ZYMV-JV | 6/6 | 436 bp |
| ZYMV-JV | | | Attenuated | |
| AGII-IFN + | AGII-IFN | ZYMV-JV | 6/6 | 955 bp |
| ZYMV-JV | | | Attenuated | |
| ZYMV-JV | No | ZYMV-JV | 2/2 | 436 bp |
| | | | Blisters, | |
| | | | mosaic, | |
| | | | filiform | |
| | | | leaves | |
| Healthy | No | No | 2/2 Healthy | None |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A nucleic acid vector for concurrently imparting herbicide resistance to a plant and cross protecting the plant, the vector comprising;
(a) sufficient potyvirus nucleic acid sequence to permit a potyvirus to replicate and spread within the plant infected by the vector;
(b) a first mutation in said potyvirus nucleic acid sequence which attenuates symptoms of said potyvirus in the plant infected by the vector;
(c) a second mutation in said potyvirus nucleic acid sequence which abolishes transmission of said potyvirus by an insect vector; and
(d) an additional nucleic acid sequence encoding a protein which imparts resistance to an herbicide when expressed in the plant infected by the vector.

2. The nucleic acid vector of claim 1, wherein said first mutation includes an amino acid substitution of the conserved FRNK box in the HC-Pro gene (SEQ ID NO.: 4) of said potyvirus nucleic acid sequence.

3. The nucleic acid vector of claim 2, wherein said amino acid substitution in the conserved FRNK box of the potyvirus nucleic acid sequence includes a substitution of a Arg to Ile at position 180 within the HC-pro gene product (SEQ ID NO.: 5) of said potyvirus.

4. The nucleic acid vector of claim 1, wherein said second mutation includes an alteration of a conserved DAG triplet at position 8 in an N terminal region of the coat protein (SEQ ID NO.: 3) of said potyvirus.

5. The nucleic acid vector of claim 4, wherein said alteration of said DAG triplet includes a substitution for an alanine residue in said DAG triplet.

6. The nucleic acid vector of claim 1, wherein said insect vector is an aphid.

7. The nucleic acid vector of claim 1, wherein said sufficient potyvirus nucleic acid sequence is derived from zucchini yellow mosaic virus (ZYMV).

8. The nucleic acid vector of claim 1, wherein said additional nucleic acid sequence is at least a portion of a phosphinothricin acetyltransferase coding sequence.

9. The nucleic acid vector of claim 1, wherein said protein is at least a functional portion of a phosphinothricin acetyltransferase.

10. A method of concurrently imparting herbicide resistance to a plant and cross protecting the plant against at least one potyvirus, the method comprising inoculating at least a portion of the plant with a vector comprising;
(a) sufficient potyvirus nucleic acid sequence to permit a potyvirus to replicate and spread within the plant infected by the vector;
(b) a first mutation in said potyvirus nucleic acid sequence which attenuates symptoms of said potyvirus in the plant infected by the vector;
(c) a second mutation in said potyvirus nucleic acid sequence which eliminates transmission of said potyvirus by an insect vector; and
(d) an additional nucleic acid sequence encoding a protein which imparts resistance to an herbicide when expressed in the plant infected by the vector.

11. The method of claim 10, wherein said first mutation includes an amino acid substitution of the conserved FRNK box in the HC-Pro gene (SEQ ID NO.: 4) of the potyvirus nucleic acid sequence.

12. The method of claim 10, wherein said amino acid substitution of the conserved FRNK box in the HC-Pro gene of the potyvirus nucleic acid sequence includes a substitution of a Arg to Ile at position 180 within the HC-pro gene product (SEQ ID NO.: 5) of said potyvirus.

13. The method of claim 10, wherein said second mutation includes an alteration of a conserved DAG triplet at position 8 in an N terminal region of the coat protein (SEQ ID NO.: 3) of said potyvirus.

14. The method of claim 13, wherein said alteration of said DAG triplet includes a substitution for an alanine residue in said DAG triplet.

15. The method of claim 10, wherein said insect vector is an aphid.

16. The method of claim 10, wherein said sufficient potyvirus nucleic acid sequence is derived from zucchini yellow mosaic virus (zymv).

17. The method of claim 10, wherein said additional nucleic acid sequence is at least a portion of a phosphinothricin acetyltransferase coding sequence.

18. The method of claim 10, wherein said protein is at least a functional portion of a phosphinothricin acetyltransferase.

19. The method of claim 10, wherein said at least a portion of a plant includes an item selected from the group consisting of at least one cell in the plant and at least one plant cell in tissue culture.

20. At least a portion of a plant treated according to the method of claim 10.

21. An infectious virion harvested from a plant treated according to the method of claim 10.


SEQUENCE LISTING (10 sequences)

SEQ ID NO: 1 - DNA, 549 bp, unknown
  phosphinothricin acetyltransferase coding sequence
atgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatc

SEQ ID NO: 2 - DNA, 837 bp, Zucchini yellow mosaic virus
tcaggcactcagccaactgtggcagacactggagctacaaagaaagataaagaagatgacaaagggaaaaacaaggacgttacaggctccggctcaggtgagaaaacagtagcagctgtcacgaaggacaaggatgtgaatgctggttctcatgggaaaattgtgccgcgtctttcgaagatcacaaagaaaatgtcattgccacgcgtgaaaggaaatgtgatactcgatattgatcatttgctggaatataaaccggatcaaattgagttatataacacacgagcgtctcatcagcagttcgcctcttggttcaaccaggttaagacggaatatgatttgaacgagcaacagatgggagttgtaatgaatggtttcatggtttggtgcattgagaatggcacttcacccgacattaatggagtgtgggttatgatggacggaaatgagcaagttgagtatcccttgaaaccaatagttgaaaatgcaaagccaacgctgcggcaaataatgcatcatttttcagatgcagcggaggcatatatagagatgagaaatgcagaggcaccatacatgccgaggtatggtttgcttcgaaacctacgggataggagtttagcacgatatgcttttgatttctatgaagtcaattctaaaactcctgaaagagcccgcgaagctgttgcgcagatgaaagcagcagctcttagcaatgtttcttcaaggttgtttggccttgatggaaatgttgccaccactagcgaagacactgaacggcacactgcacgtgatgttaatagaaacatgcacaccttactaggtgtgaatacaatgcag

SEQ ID NO: 3 - protein, 279 aa, Zucchini yellow mosaic virus
SGTQPTVADTGATKKDKEDDKGKNKDVTGSGSGEKTVAAVTKDKDVNAGSHGKIVPRLSKITKKMSLPRVKGNVILDIDHLLEYKPDQIELYNTRASHQQFASWFNQVKTEYDLNEQQMGVVMNGFMVWCIENGTSPDINGVWVMMDGNEQVEYPLKPIVENAKPTLRQIMHHFSDAAEAYIEMRNAEAPYMPRYGLLRNLRDRSLARYAFDFYEVNSKTPERAREAVAQMKAAALSNVSSRLFGLDGNVATTSEDTERHTARDVNRNMHTLLGVNTMQ

SEQ ID NO: 4 - DNA, 1359 bp, Zucchini yellow mosaic virus
tcacaaccggaagttcagttcttccaaggatggcgacgaatgtttgacaagtttaggcccagcctagatcatgtgtgcaaagttgaccacaacaacgaggaatgtggtgagttggcagcaatcttttgtcaggctctattcccagtagtgaaactatcgtgccaaacatgcagagaaaagcttagtagagttagcttcgaggaattcaaagactctttgaacgcaaactttattatccacaaggatgaatgggatagtttcaaggaaggctctcattacgataatattttcaaattgatcaaagtggcaacacaggctactcagaatctcaagctctcatctgaagttatgaagttagttcagaaccacacaagcactcacatgaagcaaatacaagacatcaacaaggcgctcatgaaaggttcattggttacgcaagacgaattggacttagctttgaaacagcttcttgaaatgactcagtggtttaagaaccacatgcatctgactggtgaggaggcattgaaaatgttcataaataagcgctctagcaaggccatgataaatcctagccttctatgtgacaaccaattggacaaaaatgaaattttgtttggggagaaagagatacattccaagcgattattcaagaacttcttcgaagaagtataccagcgaaggatatacgaagtacgtagtgcgaactttccaaatggtactcgtaagttggccataggctcattgattgtaccactcaatttggatagggcacgcactgcactacttggagagagtattgagaagaagccactcacatcagcgtgtgtctcccaacagaatggaaattatatacactcatgctgctgtgtaacgatggatgatggaaccccgatgtactcagagcttaagagcccgacgaagaggcatctagttataggagcttctggtgatccaaagtacattgatctgccagcatctgaggcagaacgcatgtatatagcaaaagaaggttattgctatctcaatattttcctcgcaatgcttgtgaatgttaatgagaacgaagcaaaggatttcaccaaaatgattcgtgatgttttgatccccatgcttgggcagtggccttcattgatggatgttgcaactgcagcatatattctaggtgtattccatcctgaaacgcgatgcgctgaattacccaggatccttgttgaccacgctacacaaaccatgcatgtcattgattcttatggatcactaactgttgggtatcacgtgctcaaggccggaactgtcaatcatttaattcagtttgcctcaaatgatatgcaaagcgagatgaaacattacagagttggc

SEQ ID NO: 5 - protein, 453 aa, Zucchini yellow mosaic virus
SQPEVQFFQGWRRMFDKFRPSLDHVCKVDHNNEECGELAAIFCQALFPVVKLSCQTCREKLSRVSFEEFKDSLNANFIIHKDEWDSFKEGSHYDNIFKLIKVATQATQNLKLSSEVMKLVQNHTSTHMKQIQDINKALMKGSLVTQDELDLALKQLLEMTQWFKNHMHLTGEEALKMFINKRSSKAMINPSLLCDNQLDKNEILFGEKEIHSKRLFKNFFEEVYQRRIYEVRSANFPNGTRKLAIGSLIVPLNLDRARTALLGESIEKKPLTSACVSQQNGNYIHSCCCVTMDDGTPMYSELKSPTKRHLVIGASGDPKYIDLPASEAERMYIAKEGYCYLNIFLAMLVNVNENEAKDFTKMIRDVLIPMLGQWPSLMDVATAAYILGVFHPETRCAELPRILVDHATQTMHVIDSYGSLTVGYHVLKAGTVNHLIQFASNDMQSEMKHYRVG

SEQ ID NO: 6 - protein, 190 aa, unknown
  phosphinothricin acetyltransferase coding sequence
MSPERRPADIRRATEADMPAVCTIVNHYIETSTVNFRTEPQEPQEWTDDLVRLRERYPWLVAEVDGEVAGIAYAGPWKARNAYDWTAESTVYVSPRHQRTGLGSTLYTHLLKSLEAQGFKSVVAVIGLPNDPSVRMHEALGYAPRGMLRAAGFKHGNWHDVGFWQLDFSLPVPPRPVLPVTEIVDTVMLQ

SEQ ID NO: 7 - DNA, 27 bp, Artificial
  synthetic oligonucleotide primer
atgctgcagatgagcccagaacgacgc

SEQ ID NO: 8 - DNA, 27 bp, Artificial
  synthetic oligonucleotide primer
agtctcgaggatctcggtgacgggcag

SEQ ID NO: 9 - DNA, 22 bp, Artificial
  synthetic oligonucleotide primer
agctccatacatagctgagaca

SEQ ID NO: 10 - DNA, 20 bp, Artificial
  synthetic oligonucleotide primer
tggttgaaccaagaggcgaa